(19) United States
(12) Patent Application Publication
(10) Pub. No.: US 2022/0016070 A1
(43) Pub. Date: Jan. 20, 2022

(54) PREMIXED, READY TO USE PHARMACEUTICAL COMPOSITIONS OF AMIODARONE

(71) Applicant: Good Health, LLC, Princeton, NJ (US)

(72) Inventor: Basavaraj Siddalingappa, Gujarat (IN)

(73) Assignee: Good Health, LLC, Princeton, NJ (US)

(21) Appl. No.: 17/376,355

(22) Filed: Jul. 15, 2021

Related U.S. Application Data
(60) Provisional application No. 63/140,344, filed on Jan. 22, 2021.

(30) Foreign Application Priority Data
Jul. 17, 2020 (IN) 202041030602

(51) Int. Cl. A61K 31/343; A61K 47/69; A61K 9/00; A61K 47/22; A61K 47/10; A61K 47/20; A61K 47/12; A61K 47/26; A61K 47/24

(52) U.S. Cl. CPC A61K 31/343; A61K 47/6951; A61K 9/0019; A61K 47/22; A61J 1/10; A61K 47/20; A61K 47/12; A61K 47/26; A61K 47/24; A61K 47/10

(57) ABSTRACT

Liquid amiodarone compositions having improved stability during storage are disclosed. The compositions include amiodarone, preferably as the HCl salt, a cyclodextrin such as hydroxy propyl β cyclodextrin and a surfactant, and have a pH of 3-5.

### CROSS-REFERENCE TO RELATED APPLICATIONS

This application claims the benefit of priority from Indian Provisional Patent Application Serial No. 202041030602, filed Jul. 17, 2020 and U.S. Provisional Patent Application Ser. No. 63/140,344 filed Jan. 22, 2021, the contents of each of which are incorporated herein by reference.

### BACKGROUND OF INVENTION

### Field of Invention

This invention relates to stable, premixed, ready to use compositions of amiodarone meant for intravenous administration. The invention also pertains to compositions, packing material and packing processes to maintain the drug product in stable condition for extended periods as compare to currently available amiodarone formulations.

Amiodarone was initially available as solution in a vial with Benzyl alcohol and Tween-80. The vial needs to reconstituted and infused immediately. In addition to adverse effects of excipients such as hypotension caused by Tween-80, adsorption of amiodarone on to plastic bags is another issue which makes product unstable after reconstitution.

Amiodarone is insoluble in water, it requires solubilization to make a solution for injection. Multiple options have been explored to increase the solubility of amiodarone for oral and intravenous administration. The solubility of amiodarone was increased by using complexation technology that uses cyclodextrins.

Nexterone is premixed, ready to infuse solution of Amiodarone formulated with sulfobutylether-7-beta-cyclodextrin (SAECD) that comes in special multilayer galaxy bags with shelf life of 24 months. However, the cost of the components of cyclodextrin SAECD and bags make the product costly.

Stable, ready to infuse solutions, cost efficient formulations of amiodarone are needed. Hence this invention describes simple, ready to infuse solution formulations of amiodarone without using SAECD.

U.S. Pat. No. 6,869,939 describes amiodarone formulations comprising of SAECD wherein the ratio of CD to drug is >1.1. The solutions are said to be stable at room temperature with significant precipitation.

PCT/US03/13250 describes compositions of amiodarone with SAECD with a ratio of CD to drug of >1.1. The formulations are said to be without significant precipitation at room temperature.

U.S. Pat. No. 7,635,773 describes a pure form of a sulfoalkyl cyclodextrin having lesser amounts of a drug degrading impurity.

Jacobs, M. S., Luinstra, M., Moes, J. R., Chan, T. C., Minovic, I., Frijlink, H. W., & Woerdenbag, H. J. (2017. European Journal of Hospital Pharmacy, 24(2), 110-114 describe ready to use parenteral formulations of amiodarone with both sulfa butyl and hydroxyl propyl cyclodextrins. The authors conclude that stable formulations of amiodarone were possible with lesser SAECD compared to HPβCD.

Cushing, D. J., Adams, M. P., Cooper, W. D., Kowey, P. R., & Lipicky, R. J. (2009) describe the bioequivalence of 2 intravenous amiodarone formulations in healthy participants in The Journal of Clinical Pharmacology, 49(4), 407-415. This paper compares the pharmacokinetic profile of amiodarone formulations and concludes that formulations with SAECD are bioequivalent to diluted benzyl alcohol/Tween containing formulations. Several advantages of SAECD formulations are listed.

Most of the above-mentioned patents describe formulations of amiodarone with SAECD. However, formulations with low amount of HPβCD are not described.

The amiodarone formulations with HPβCD often tend to precipitate at room temperature and may rapidly precipitate when diluted with buffer having pH 7.4.

Hence amiodarone formulations with HPβCD which do not precipitate at room temperature and hold drug from immediately precipitating after diluting with buffer pH 7.4 are needed.

### SUMMARY OF THE INVENTION

The invention relates to liquid, ready to infuse, formulations of amiodarone in plastic infusion bags.

This invention also describes methods of treating of patients in need of treatment with the compositions of the invention. Such methods include administering a drug-containing composition as described herein to a patient in need of such drug, preferably by parenterally administering the composition without further reconstitution or transformation of the formulation in the infusion bag prior to administration.

The invention also relates to methods of making and methods of packing compositions of this invention.

### BRIEF DESCRIPTION OF THE DRAWINGS

FIG. 1 is a graph plotting the concentration of HPβCD vs amiodarone solubility, corresponding to Example 5.

FIG. 2 is a graph plotting the concentration of HPβCD vs amiodarone solubility (moles) in presence of 0.36 mg/mL of DSPE-PEG2000, Na, corresponding to Example 5.

FIG. 3 is a graph plotting the concentration of HPβCD vs amiodarone solubility (moles) in presence of 0.36 mg/mL of Vitamin E TPGS, corresponding to Example 5.

### DETAILED DESCRIPTION OF THE INVENTION

The invention relates to stable, liquid pharmaceutical compositions comprising amiodarone, a cyclodextrin and a surfactant. The liquid amiodarone compositions of the invention preferably have a pH of about 3.0-5.0 and have extended stability, particularly when they are stored in a pharmaceutically-acceptable containers in combination with an amount of an inert gas, at temperature of from about 5-25° C. for a period of at least about 6 months, with the amiodarone retaining at least about 90% of its starting concentration.

The compositions of this inventions are preferably packed in plastic infusion bags and include amiodarone, an aqueous vehicle such as water for injection, a solubilizer such as cyclodextrin, and a surfactant. Also preferably included are pharmaceutically acceptable ancillary ingredients including but not limited to a tonicity agent, such as dextrose, other excipients such as a surfactant, a pH adjustor, a preservative, and other ancillary excipients which will be apparent to those of ordinary skill. The pH of the compositions in the plastic bags will preferably be from at least about 3.4 to about 4.5. The drug-containing compositions of the invention are preferably stored in suitable plastic packaging along with an amount of an inert gas such as nitrogen, the nitrogen being used as a blanketing gas. Plastic bags made from non-polar polymers such as PP INERTA, GALAXY®, EXCEL®, VISIV®, and VIAFLO can be used for storing of amiodarone solution of this invention. Without limitation, suitable bags can be made from materials such as polypropylene, polyvinylchloride, polyethylene and ethyl vinyl acetate or any other compatible polymeric material. In another aspect, suitable bags comprise a copolyester, polyethylene or polyolefin, etc. surface in contact with the liquid amiodarone composition.

A “stable” composition of the invention means a pharmaceutical composition having sufficient stability at room temperature conditions to have utility as a pharmaceutical product. Preferably, a “stable” composition of the invention has sufficient stability to allow storage at room temperature conditions, preferably between about 15° C. and about 30° C., more preferably about 20° C. to about 25° C., most preferably about 25° C., and between about 55% to about 65% RH (e.g., about 60% RH), for a reasonable period of time, e.g., the shelf-life of the product which can be as short as one month but is typically six months or longer, more preferably one year or two years. A “stable” composition of the invention also includes specific ranges of impurities as described herein. Preferably, a “stable” composition is one which has minimal degradation of the at least one drug, e.g., it retains at least about 90% of un-degraded active, preferably at least about 95%, more preferably at least about 99%, after storage at about 15-30° C. for a 1 to 3 year period of time.

As can be seen in the examples below, the inventive compositions have extended stability when measured at 6 months at 40 C/75% RH. This corresponds to at least 2 years at room temperature conditions, e.g. 25 C/60% RH. An additional example demonstrates even greater stability over 8 months @25 C/60% RH. It is therefore possible to provide amiodarone compositions which demonstrate the quality of being stable when stored at a pH of 3.0-5.0 in a pharmaceutically-acceptable container in combination with an amount of an inert gas, at temperature of from about 5-25° C. for a period of at least about 6 months, with the amiodarone retaining at least about 90% of its starting concentration.

The invention also relates to stable, liquid formulations of pharmaceutical compositions comprising amiodarone or a pharmaceutically acceptable salt, ester, or prodrug thereof. These amiodarone compositions are stable at room temperature conditions (25° C./60% RH) for extended periods of time (e.g., stable for >6 months, preferably >1 year, more preferably >2 years, with impurities less than or equal to acceptable limits (e.g., total impurities <3%, preferably <2%, more preferably <1%, as determined by HPLC after storing formulation at 40° C./75% RH for about 3 months).

Preferably, the amiodarone is in its native form or its pharmaceutically acceptable salt, ester, or prodrug thereof (e.g., amiodarone HCl). The term “amiodarone” includes amiodarone or a pharmaceutically acceptable salt, ester, or prodrug thereof. The amiodarone may alternatively be in the form of a complex. The amiodarone may be present in the compositions of the invention in any amount, such as an amount ranging from about 0.05 mg/mL to about 10 mg/mL, or alternatively an amount of from about 0.1 mg/mL to about 2.5 mg/mL, or, in some embodiments, an amount of from about 1 mg/mL to about 2 mg/mL such as 1.5 mg/mL or 1.8 mg/mL.

The amiodarone compositions of this embodiment comprise of at least one cyclodextrin which is preferably a freely water soluble form of cyclodextrin such as Hydroxy propyl-β-cyclodextrin (HPβC). The amount of cyclodextrin included in the amiodarone compositions of the invention can be from about 0.5% to about 5% W/V of composition, with amounts of about 1.5 to about 3% W/V or 2 to 3% W/V or about 2.5 being preferred in some aspects of the invention.

The amiodarone compositions of this invention additionally contain a safe, non-immunogenic, non-hypersensitive surfactant, such as and without limitation a phospholipid, PEG-Cholesterol, PEG-Vitamin E (i.e., Vitamin E TPGS), Cremophore, Tweens, solutols and others.

Two preferred surfactants are Pegylated phospholipids such as 1,2-distearoyl-sn-glycero-3-phosphoethanolamine-N-[amino(polyethylene glycol)-2000] Na, DSPE-PEG2000 and Vitamin E TPGS.

The amount of surfactant included in the compositions can range from about 0.1% W/V to about 0.5% W/V, or from about 0.2 to about 0.5%, or more preferably, from about 0.3 to about 0.4% W/V.

The amiodarone compositions preferably have pH value of at least 3.4. Alternatively, in some embodiments the pH is in the range of between 2.9-5.5, and is preferably, between 3.5-5.0.

The amiodarone compositions may also contain one or more buffering agents, such as but not limited to sodium citrate, sodium acetate, sodium phosphate. The buffering agents will be in the compositions at a concentration of 0.05 mg/mL to 5 mg/mL, more preferably 0.1 to 3 mg/mL. most preferably 0.1 to 0.5 mg/mL.

The compositions of the invention may contain acid for pH adjustment and to induce solubilization of the amiodarone. The acidic excipients may include but are not limited to citric acid, succinic acid, methyl sulfonic acid, malic acid, malonic acid, maleic acid, HCl, H₂SO₄, gluconic acid, lactic acid and lactobionic acid. The amount acidic excipient in the composition range between about 0.01 to about 1% W/V or from about 0.01 to about to 0.5% W/V of the composition.

Amiodarone compositions of this embodiment may contain excipients for tonicity adjustment. Tonicity adjusting agents can be for example sodium chloride, glycerol, propylene glycol, dextrose, lactose, mannitol, sorbitol, sucrose. Suitable amounts may be from about 0.1 mg/mL to about 60 mg/mL, more preferably from about 0.6 to about 55 mg/mL, most preferably from about 0.8 to about 55 mg/mL.

Compositions of the embodiments may also contain preservatives, antioxidants, chelating agents.

The preferred antioxidants for the amiodarone compositions described herein are sodium meta bisulfate, BHA. BHT, monothioglycerol, Vitamin E and its esters.

### EXAMPLES

### Materials:

Amiodarone HCl (>99% purity) was procured from TCI Chemicals (India) Pvt. Ltd. Phosphatidyl Choline (Soya) and 1, 2-Distearoyl-sn-glycero-3-phosphoethanolamine-Poly(ethylene glycol) (DSPE-PEG2000) were procured from Lipoid GMBH, Germany. Propylene Glycol AR grade was procured from Merck Lifesciences India Pvt Ltd, Hydroxy propyl-β-cyclodextrin (Kleptose HPB) was purchased for Signet Chemicals, India.

Polypropylene infusion bags (Inerta) were procured from Technoflex, Germany. All other chemicals and reagents were sourced locally were used without further purification.

### Methods:

### HPLC Method:

The method reported in USP for determination of assay of Amiodarone injection was used for assessing potency during stability testing of samples of Amiodarone. Method is as described below.

### Preparation of Mobile Phase:

Buffer: 6.8 g of monobasic potassium phosphate in 900 mL water, add 1 mL of Triethylamine, adjust pH to 6.0±0.5 and make up volume to 1000 mL mix well.

Mobile phase: Mix Acetonitrile 500 mL and buffer 500 mL in glass bottle and filter through 0.44 micron nylon filter.

Diluent: 500 mL of buffer was mixed with 500 mL of Acetonitrile.

### Preparation of Standard Solution:

0.1 mg/mL of Amiodarone in diluent.

### Sample Preparation:

Appropriate volume of sample diluted with diluent to get nominal concentration of 0.1 mg/mL of Amiodarone.

### Chromatographic Conditions:

Flow rate: 1.5 mL/min,

Column: Grace: 3.9-mm 15-cm; 5-μm packing L26.
Column temperature: 40° C.
Injection volume: 10 μL

### Detection: 240 nm

### Example 1: Formulations with HPβCD with Different Acidic Excipients for pH Adjustment

TABLE 1
Compositions with HPβCD

| | Form-1 | Form-2 | Form-3 |
|---|---|---|---|
| Ingredients | Quantity | Quantity | Quantity |
| Amiodarone HCl | 200 mg | 200 mg | 180 mg |
| 10% Methane Sulfonic acid | 0 | Few drops | 0 |
| Poloxamer 188 | 0 | 0 | 3.0 mg |
| HPβCD (Kleptose HPB) | 1000 mg | 1000 mg | 1000 mg |
| Succinic acid | 100 mg | 0 | 100 mg |
| Distilled water | Qs 100 mL | Qs 100 mL | Qs 100 mL |
| pH | 3.14 | 3.5 | 3.1 |
| Storage | In PP bag, | In PP bag, | In PP bag, |
| | 100 mL | 100 mL | 100 mL |

### Preparation:

Step 1: Cyclodextrin was dissolved in water.

Step 2: Amiodarone was added to step-1 followed by specified acidic excipient or solutions, mixed for 2-3 minutes and heated at 50-60° C. until all the Amiodarone was dissolved.
Step 3: After cooling step-2 mixture, other ingredients were added and mixed at room temperature. The solutions were filtered through 0.22 micron nylon filter.

### Packing:

About 50 mL of above formulations were filled into 100 mL polypropylene bags. The rest of the volume of the bags was filled with Nitrogen. Bags were little bulged.

TABLE 2
Stability data for Form-1-3

| | % Assay | | |
|---|---|---|---|
| | | 40° C./75% RH, | 40° C./75% RH, |
| Formulation | Initial | 1 month, 15 days | 4 month, 25 days |
| F-1 | 100.21 | 97.6 | 95.9 (pH 3.35) |
| F-2 | 106.85 | 101.7 | 104.54 (pH 3.16) |
| F-3 | 106.23 | 98.68 | 99.79 (pH 3.21) |

Observations:
1. Formulations 1-3 are stable at 40 C/75% for at least 4 months.
2. At 25° C./60% RH, some fiber-like particles are observed after 4 months.
3. The formulations 1-3 when diluted 1:10 with phosphate buffer pH 7.4, rapid precipitation was observed. This would pose a problem, as rapid precipitation at site injection due to higher blood pH would cause pain at site of injection.

### Example 2: Formulations with Phospholipids

TABLE 3
Amiodarone compositions with phospholipids

| Ingredients | Form 4 | Form 5 | Form 6 | Form 7 | Form 8 | Form 9 | Form 10 | Form 11 |
|---|---|---|---|---|---|---|---|---|
| Amiodarone HC1 | 180 mg | 180 mg | 180 mg | 180 mg | 180 mg | 180 mg | 180 mg | 180 mg |
| Sod. Benzoate | 100 mg | 100 mg | — | — | — | — | 100 mg | 100 mg |
| HP CD | 1000 mg | 1000 mg | 1000 mg | 1000 mg | 1000 mg | 1000 mg | 1000 mg | 1000 mg |
| (Kleptose HPB) | | | | | | | | |
| DSPE-PEG2000, Na | 16.5 mg | 16.5 mg | 16.5 mg | 16.5 mg | 25 mg | 25 mg | 25 mg | 25 mg |
| Dextrose | 4 gm | 4.5 gm | 5 gm | 4.5 gm | 5 gm | 4.7 gm | 4.0 gm | 4.6 gm |
| 10% Succinic acid | 5 mL | — | 0.9 mL | — | 0.9 mL | — | 4.3 mL | — |
| 2% MSA | — | 2.2 mL | — | 1.1 mL | — | 0.6 mL | — | 2.6 mL |
| Purified water | 100 mL | 100 mL | 100 mL | 100 mL | 100 mL | 100 mL | 100 mL | 100 mL |
| pH | 3.45 | 3.40 | 3.45 | 3.52 | 3.54 | 3.47 | 3.47 | 3.45 |
| Osmolarity | 295 | 292 | 305 | 281 | 295 | 286 | 283 | 284 |

Process for Formulation F-4 to F-11.

Step 1: Cyclodextrin was dissolved in water.

Step 2: Amiodarone was added to step-1 followed by specified acidic excipient or solutions, pH adjusted to desired value per table mixed for 2-3 minutes and heated at 50-60° C. till all the Amiodarone was dissolved.

Step 3: After cooling step-2 mixture, other ingredients were added and mixed at room temperature. The solutions were filtered through 0.22 micron nylon filter.

Packing: 50 mL solution in 100 mL PP bag with rest of the volume nitrogen.

Dilution Testing with Phosphate Buffer pH 7.4

The blood has pH approximately 7.4, if the formulations precipitate out immediately after dilution with buffer pH 7.4, this indicates a potential problem. However slow or precipitation after some time would avoid problem as drug gets bonded to plasma protein or undergoes infinite dilution in blood within about 5 minutes. All the formulations F-4 to F-11 were diluted and observed for precipitation in comparison with RLD (Nexterone).

TABLE 4
Observations of Form-4 to Form-11 after dilution with buffer pH 7.4

| | | Obser- | Observation |
|---|---|---|---|
| Formu- | | vation | (After |
| lation | Mixing | (Initial) | 15-20 min) |
| RLD | 1 mL formulation + | Slightly | More turbid |
| | 10 mL phosphate buffer pH-7.4 | turbid | or ppt |
| Form-4 | 1 mL formulation + | Slightly | Same as RLD |
| | 10 mL phosphate buffer pH-7.4 | turbid | |
| Form-5 | 1 mL formulation + | Slightly | Less turbid |
| | 10 mL phosphate buffer pH-7.4 | hazy | than RLD |
| Form-6 | 1 mL formulation + | Slightly | Same as RLD |
| | 10 mL phosphate buffer pH-7.4 | hazy | |
| Form-7 | 1 mL formulation + | Slightly | Less turbid |
| | 10 mL phosphate buffer pH-7.4 | hazy | than RLD |
| Form-8 | 1 mL formulation + | Slightly | Less turbid |
| | 10 mL phosphate buffer pH-7.4 | hazy | than RLD |
| Form-9 | 1 mL formulation + | Slightly | Less turbid |
| | 10 mL phosphate buffer pH-7.4 | hazy | than RLD |
| Form-10 | 1 mL formulation + | Slightly | Same as RLD |
| | 10 mL phosphate buffer pH-7.4 | hazy | |
| Form-11 | 1 mL formulation + | Slightly | Less turbid |
| | 10 mL phosphate buffer pH-7.4 | hazy | than RLD |

### Observations:

Formulations F-4 to F-11 were either comparable to RLD or better than RLD in terms of sustaining precipitation of Amiodarone after dilution with buffer pH 7.4. Inclusion of DSPE-PEG has significant effect on retarding precipitation.

TABLE 5
Stability data of Formulations F-4 to F11

| | Formulation numbers | | | | | | | |
|---|---|---|---|---|---|---|---|---|
| Assay (%) | F-4 | F-5 | F-6 | F-7 | F-8 | F-9 | F-10 | F-11 |
| Initial | 92.05 | 94.48 | 98.02 | 99.75 | 97.83 | 92.43 | 97.26 | 95.95 |
| 2 months at | 87.78 | 94.20 | 91.99 | 90.84 | 91.64 | 93.52 | 83.56 | 91.72 |
| 40° C./75% RH, | | | | | | | | |
| against initial | | | | | | | | |
| 3 months at | 78.77 | 84.77 | 83.27 | 83.53 | 83.52 | 91.78 | 78.62 | 86.20 |
| 40° C./75% RH, | | | | | | | | |
| against initial | | | | | | | | |
| pH after 3 | 3.25 | 3.01 | 3.04 | 2.96 | 2.98 | 3.10 | 3.45 | 3.03 |
| months at | | | | | | | | |
| 40° C./75% RH | | | | | | | | |

Observations:

1. Stability data indicate that sodium benzoate is not helpful stabilizing amiodarone.
2. Formulations with methane sulfonic acid as pH adjusting agent are better in stability.
3. Form-9 is stable compared to other formulations.
4. Decrease in potency probably due to adsorption.
5. Decrease in pH observed over time.
6. Form-4 to F-11 remain clear at 25° C./60% RH.

### Example 3: Formulations with DSPE-PEG and Buffering Agent

TABLE 6
Formulations F-12 and F-13 with stability data

| Ingredients | Form 12 | | Form 13 | |
|---|---|---|---|---|
| Amiodarone HCl | 180 | mg | 180 | mg |
| HPβCD | 2.5 | g | 2.5 | g |
| (Kleptose HPB) | | | | |
| DSPE-PEG2000, Na | 36 | mg | 36 | mg |
| Dextrose | 4.1 | gm | 4.15 | gm |
| Sodium citrate | 18.3 | mg | 18.3 | mg |
| Citric acid anhydrous | 36.2 | mg | 36.2 | mg |
| Sodium Metabisulfite | 5 | mg | 5 | mg |

| | | | |
|---|---|---|---|
| 10% Citric acid | 0.1 | mL | — |

| | | | |
|---|---|---|---|
| 10% Succinic acid | — | 0.4 | mL |

| | | |
|---|---|---|
| Purified water | Up to 100 mL | Up to 100 mL |

Process and packaging for F-12 and F-13 was same as F-4 to F-11

TABLE 7
Stability data for Form-12

| | | | | | | Stability |
|---|---|---|---|---|---|---|
| | | | | | | condition: |
| | | | | | | 25° C./ |
| | | | | | | 60% RH |
| | Stability condition: 40° C./75% RH (Months) | | | | | (Months) |
| Test | 1 | 2 | 3 | 4 | 6 | 8 |
| Description | Clear | Clear | Clear | Clear | Clear | Clear |
| | colorless | colorless | colorless | colorless | colorless | colorless |
| | solution | solution | solution | solution | solution | solution |
| pH | 3.35 | 3.62 | 3.44 | 3.35 | 3.35 | 3.46 |
| % Assay | 97.5 | 95.9 | 98.8 | 99.0 | 104.9 | 297 |
| Impurities | % Impurity | % Impurity | % Impurity | % Impurity | % Impurity | % Impurity |
| Related | 0.12 | 0.14 | 0.05 | 0.10 | 0.087 | 0.06 |
| compound-D | | | | | | |
| Related | 0.01 | 0.03 | 0.02 | 0.01 | 0.013 | 0.03 |
| compound-E | | | | | | |
| Any unspecified degradation product listed by RRT (%) | | | | | | |
| 0.230 | ND | 0.03 | ND | ND | ND | ND |
| 0.240 | 0.03 | ND | ND | ND | ND | 0.041 |
| 0.26 | ND | ND | 0.05 | 0.04 | 0.04 | ND |
| 0.291 | 0.05 | ND | ND | ND | ND | ND |
| 0.298 | ND | 0.06 | ND | ND | ND | 0.065 |
| 0.350 | ND | ND | 0.02 | ND | ND | ND |
| 0.376 | 0.04 | ND | ND | ND | ND | ND |
| 0.400 | ND | ND | ND | ND | 0.029 | ND |
| 0.410 | ND | 0.03 | ND | 0.02 | ND | 0.007 |
| 0.443 | 0.02 | ND | ND | ND | ND | |
| 0.470 | ND | ND | 0.02 | ND | ND | 0.014 |
| 0.477 | ND | 0.03 | ND | ND | ND | ND |
| 0.563 | 0.05 | ND | ND | ND | ND | ND |
| 0.560 | ND | ND | 0.05 | 0.05 | 0.05 | ND |
| 0.576 | ND | 0.05 | ND | ND | ND | 0.024 |
| 0.700 | ND | ND | ND | ND | 0.022 | 0.035 |
| 1.15 | ND | ND | 0.08 | ND | ND | ND |
| 1.17 | ND | ND | ND | 0.02 | ND | ND |
| 1.30 | ND | ND | ND | ND | 0.14 | ND |
| Total | 0.35 | 0.51 | 0.39 | 0.31 | 0.29 | 0.284 |

TABLE 8
Stability data for Form-13

| | Stability time points | | | | |
|---|---|---|---|---|---|
| | | 1M 5D | 1M 20D | 3M | 4M 13D |
| Parameters | Initial | @40 C./75% RH | @40 C./75% RH | @40 C./75% RH | @40 C./75% RH |
| pH | 3.37 | 3.25 | 3.54 | 3.38 | 3.26 |
| Osmolality | 277 | Not Analyzed | Not Analyzed | Not Analyzed | Not Analyzed |
| Assay (%) | 99.25 | 97.42 | 98.31 | 104.51 | 105.46 |
| Related | Not | 0.01@RRT0.408 | 0.03@RRT0.385 | 0.02@RRT0.35 | 0.01@RRT0.38 |
| compound-E (%) | analyzed | | | | |
| Related | Not | 0.09@RRT0.513 | 0.09@RRT0.542 | 0.08@RRT0.502 | 0.07@RRT0.49 |
| compound-D (%) | analyzed | | | | |
| Any unspecified | Not | 0.03@RRT0.24 | 0.01@RRT0.199 | 0.04@RRT0.26 | 0.04@RRT0.26 |
| impurities (%) | analyzed | 0.05@RRT0.29 | 0.03@RRT0.23 | 0.09@RRT0.30 | 0.01@RRT0.29 |
| | | 0.04@RRT0.37 | 0.06@RRT0.29 | 0.08@RRT0.56 | 0.01@RRT0.30 |
| | | 0.01@RRT0.44 | 0.01@RRT0.34 | 0.06@RRT1.15 | 0.02@RRT0.41 |
| | | 0.01@RRT0.46 | 0.02@RRT0.41 | | 0.07@RRT0.56 |
| | | 0.07@RRT0.563 | 0.07@RRT0.57 | | 0.01@RRT0.68 |
| | | 0.01@RRT0.68 | 0.01@RRT0.68 | | |
| | | 0.01@RRT0.849 | 0.01@RRT0.85 | | |
| | | 0.13@RRT1.23 | 0.09@RRT1.32 | | |
| Total impurities | — | 0.46% | 0.44% | 0.37% | 0.24 |

### Results:

Form-12 and Form-13 were found to have better stability when compared to formulations F-4 to F-11. The pH did not change drastically after storage at 40° C./75% RH for 6 months. Potency remained unchanged. However, F-12 is relatively better over F-13 in terms maintaining pH and potency.

Form-12 and F-13 remain clear at 25° C./60% RH after six months against reported precipitation of drug with formulation containing Hydroxy-Propyl-β-Cyclodextrin.

### Example 4: Formulations with Vitamin E TPGS

TABLE 9
Composition for formulations with Vitamin E TPGS

| | Form-14 | | Form-15 | |
|---|---|---|---|---|
| | Qty | Qty | Qty | Qty |
| Ingredients | (gm/Batch) | (mg/mL) | (gm/Batch) | (mg/mL) |
| Amiodarone | 3.6 | 1.8 | 3.6 | 1.8 |
| HPβCD | 50 | 25 | 40 | 20 |
| Vitamin-E-TPGS | 0.720 | 0.36 | 0.720 | 0.36 |
| Citric acid | 0.724 | 0.362 | 0.724 | 0.362 |
| anhydrous | | | | |
| Tri-sodium citrate | 0.366 | 0.183 | 0.366 | 0.183 |
| dehydrate | | | | |
| Dextrose | 87 | 43.5 | 90 | 45 |
| anhydrous | | | | |
| Distilled water | Up to | Up to | Up to | Up to |
| | 2000 mL | 1 mL | 2000 mL | 1 mL |
| 10% Citric acid | Q.S. | Q.S. | Q.S. | Q.S. |

| | | |
|---|---|---|
| pH | 3.454 | 3.455 |
| Osmolarity | +285 | +288 |

Process: same as Form-12 and Form-13, Vitamin E TPGS replaced DSPE-PEG, 2000.

TABLE 10
Stability data of Form-14 and Form-15

| | Form-14 | | | Form-15 | | |
|---|---|---|---|---|---|---|
| | | | 2 months | | | 2 months |
| | | | (1 Month, | | | (1 Month, |
| | | 1 Month | 25 days) | | 1 Month | 25 days) |
| | | 40° C./ | 40° C./ | | 40° C./ | 40° C./ |
| Test Details | Initial | 75% RH | 75% RH | Initial | 75% RH | 75% RH |
| Description | Clear | Clear | Clear | Clear | Clear | Clear |
| | Colorless | Colorless | Colorless | Colorless | Colorless | Colorless |
| | Solution | Solution | Solution | Solution | Solution | Solution |
| pH | 3.437 | 3.494 | 3.526 | 3.217 | 3.199 | 3.293 |
| Osmolality | 285 | 291 | 291 | 288 | 296 | 295 |
| Assay | 97.99 | 96.92 | 97.45 | 99.14 | 96.58 | 98.21 |
| AMD Rel | | | | | | |
| Comp D | 0.01 | 0.04 | 0.04 | 0.01 | 0.03 | 0.04 |
| AMD Rel | 0.02 | 0.03 | 0.03 | 0.02 | 0.02 | 0.03 |
| Comp E | | | | | | |
| RRT @ 0.24 | 0.03 | 0.03 | 0.03 | 0.03 | 0.03 | 0.03 |
| RRT @ 0.29 | 0.03 | 0.04 | 0.05 | 0.03 | 0.04 | 0.05 |
| RRT @ 0.38 | 0.01 | 0.01 | 0.01 | 0.01 | 0.01 | 0.01 |
| RRT @ 0.57 | 0.01 | 0.01 | 0.01 | — | 0.01 | 0.01 |
| RRT @ 0.65 | — | 0.01 | 0.01 | — | 0.01 | 0.01 |
| RRT @ 0.70 | 0.01 | 0.01 | 0.01 | 0.01 | — | — |
| RRT @ 0.85 | 0.01 | — | — | 0.01 | — | — |
| Total | 0.13 | 0.18 | 0.19 | 0.12 | 0.15 | 0.18 |
| Impurities | | | | | | |

TABLE 11
Observations of Form-14 and Form-15 after dilution with buffer pH 7.4

| | | Obser- | Observation |
|---|---|---|---|
| Formu- | | vation | (After |
| lation | Mixing | (Initial) | 15-20 min) |
| RLD | 1 mL formulation + | Slightly | More turbid |
| | 10 mL phosphate buffer pH-7.4 | turbid | or ppt |
| Form-14 | 1 mL formulation + | Clear | Slightly turbid |
| | 10 mL phosphate buffer pH-7.4 | solution | |
| Form-15 | 1 mL formulation + | Clear | Slightly turbid |
| | 10 mL phosphate buffer pH-7.4 | solution | |

Inferences:
Form-14 and Form-15 are better in terms of stability. Replacing DSPE-PEG with Vitamin E TPGS in the formulation did not affect chemical stability or physical stability (Clarity).
Precipitation studies after dilution of formulations with buffer pH 7.4 revealed that Form-14 and 15 withstood precipitation and remained clear for about 8-10 minutes, whereas RLD turns slightly hazy immediately after dilution and turns very turbid after about 15 minutes.
Vitamin E TPGS containing formulations lack risk of precipitation at blood pH at site of injection. Hence better patient compliance.

### Example 5: Phase Solubility Studies of Amiodarone in Presence of Various Concentration of HPβCD with and without Surfactants

Procedure:

Phase solubility studies without surfactants:
1. Acetate buffer pH 4.0 was prepared.
2. The HPβCD was added to pH-4.0 buffer concentration of 0 to 20% W/V or 0 to 0.142 Moles/L.
3. Amiodarone, 200 mg/mL was added above buffer solution with varying concentration of cyclodextrin.
4. Resulting mixtures were shaken for 24 hours at 28° C. Solutions were filtered analyzed by HPLC after suitable dilutions.
   Phase Solubility Studies with Surfactants:
1. Acetate buffer pH 4.0 was prepared, 0.36 mg/mL of either DSPE or Vitamin ETPGS was added to buffer and dissolved.
2. The HPβCD was added to pH-4.0 buffer concentration of 0 to 20% W/V or 0 to 0.142 Moles/L.
3. Amiodarone, 200 mg/mL was added above buffer solution with varying concentration of cyclodextrin.
4. Resulting mixtures were shaken for 24 hours at 28° C. Solutions were filtered analyzed by HPLC after suitable dilutions.

Results:

TABLE 12
Phase Solubility study of Amiodarone with HPβCD

| No. | Molar Conc. of HPβCD | Solubility of Amiodarone (Mols/Liter) |
|---|---|---|
| 1 | 0.0 (S0) | 0.003974 |
| 2 | 0.008929 | 0.004149 |
| 3 | 0.017857 | 0.00594 |
| 4 | 0.035714 | 0.009482 |
| 5 | 0.071429 | 0.014038 |
| 6 | 0.142857 | 0.020348 |

TABLE 13
Phase Solubility study of Amiodarone with HPβCD and DSPE

| | Molar Conc. of HPβCD | |
|---|---|---|
| | with DSPE-PEG | Solubility of |
| No. | 2000. Na, 0.36 mg/mL | Amiodarone (Mols/Liter) |
| 1 | 0.0 (S0) | 0.004745 |
| 2 | 0.008929 | 0.004592 |
| 3 | 0.017857 | 0.006292 |
| 4 | 0.035714 | 0.009494 |
| 5 | 0.071429 | 0.014238 |
| 6 | 0.142857 | 0.020698 |

TABLE 14
Phase Solubility study of Amiodarone with HPβCD and Vitamin E TPGS

| | Molar Conc. of HPβCD | |
|---|---|---|
| | with Vitamin E | Solubility of |
| No. | TPGS, 0.36 mg/mL | Amiodarone (Mols/Liter) |
| 1 | 0.0 (S0) | 0.003464 |
| 2 | 0.0089 | 0.004573 |
| 3 | 0.0179 | 0.00692 |
| 4 | 0.0357 | 0.009952 |
| 5 | 0.0714 | 0.014356 |
| 6 | 0.1429 | 0.021195 |

TABLE 15
Binding constants of Amiodarone with HPβCD

| | | | | Binding Constant, |
|---|---|---|---|---|
| Slope | S0 | (1-Slope) | S0 (1-Slope) | M⁻¹ (Kapp) |
| 0.1184 | 0.003974 | 0.8816 | 0.003503478 | 33.794985 |
| 0.1184 | 0.004745 | 0.8816 | 0.004182751 | 28.306728 |
| 0.1195 | 0.003464 | 0.8805 | 0.003050047 | 39.179721 |

Note: Binding constant calculated using formula

$$Kapp = \frac{Slope}{So ( 1 - Slope )} ,$$

So is solubility of Amiodarone buffer without cyclodextrin.

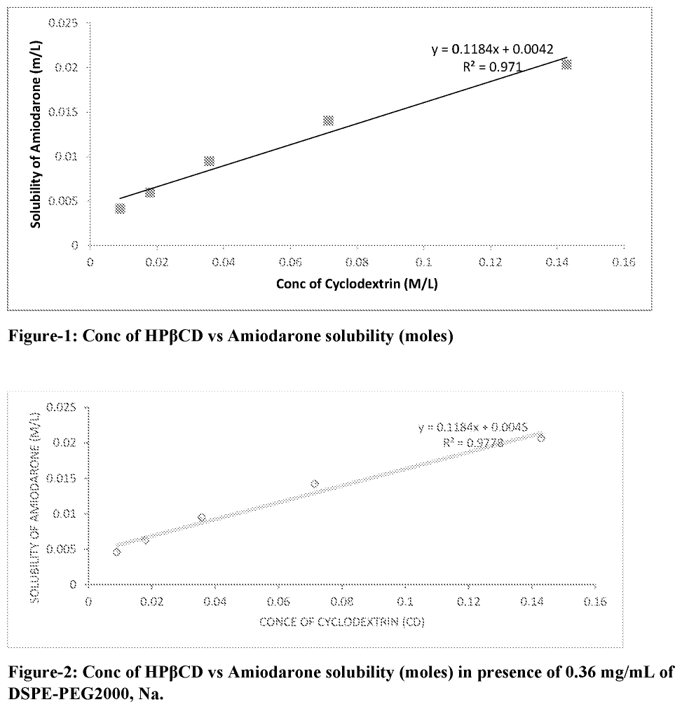

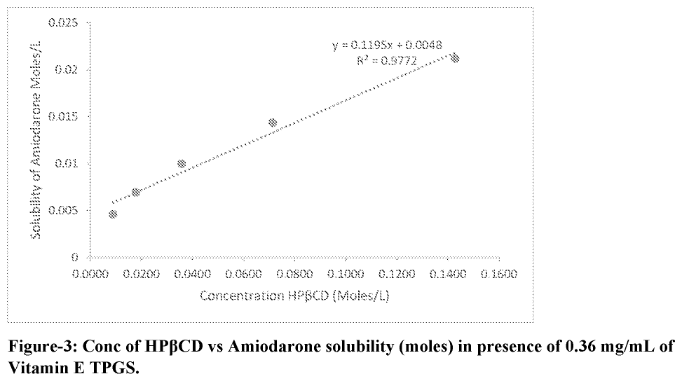

Turning now to FIGS. 1-3, it can be seen that Cyclodextrin solubilize the drug through complexation with drugs. In most of cases, solubility of drug would produce linearity relationship with concentration, solubility increases as the concentration of Cyclodextrin increases. Binding constant is usually derived from slope of solubility curve and solubility of drug with “0” cyclodextrin.

Auxiliary excipients such as acids, bases, polymers and surfactants are included along with cyclodextrins to produce greater solubility. While the auxiliary excipients do help the formulations by increasing complexation efficiency (e.g. an increase in drug solubility per given quantity of cyclodextrin) or by increasing binding constant.

The increased complexation efficiency helps improve bioavailability, and increased drug loading in the liquid formulation. Other hand, increased binding constant helps avoid decomplexation and drug precipitation.

In case of amiodarone, surfactants such as DSPE-PEG and Vitamin E TPGS neither increase complexation efficiency nor binding constant, yet surprisingly avoid precipitation after diluting with buffer pH 7.4.

Inferences:
1. Phase solubility studies indicated that solubility profile remains same with and without cyclodextrin.
2. There is no synergistic effect to surfactants on solubility or binding constant with cyclodextrins.
3. However, addition of surfactants such as DSPE-PEG or Vitamin E TPGS help resist precipitation. It is quite surprising observations, despite no synergy, surfactants do help in resisting precipitation and Vitamin E TPGS is better.

Overall Observations:
The amiodarone has poor solubility in water.
HPβCD solubilizes amiodarone at acidic pH between 3.0-4.0.
Compositions with only HPβCD precipitate rapidly after dilution with buffer pH 7.4 at 1:10 ratio. This would cause pain at site of injection and blockade of vein.
Inclusion of DSPE-PEG significantly increases sustainability of composition in preventing precipitation of amiodarone after dilution with buffer pH 7.4.
The inclusion of Vitamin E TPGS in the composition clearly avoids precipitation after dilution with buffer pH 7.4 and is better than DSPE-PEG amiodarone formulations (but still acceptably improved) and RLD (reference listed drug).
Surprisingly, the inclusion of DSPE-PEG/TPGS in the formulations resulted in multiple benefits such as prevention of rapid precipitation after dilution with buffer pH 7.4, decreases loss of potency due to adsorption, and prevents the development of haziness or precipitation while stored at 25° C./60% RH.
Despite no synergistic effect of the surfactants on the solubility of amiodarone in presence of various concentration of cyclodextrin, precipitation after dilution with pH 7.4 was prevented.
Above effect cannot be explained based on previously suggested synergy in solubilization. It is quite surprising.

1. A liquid amiodarone composition suitable for parenteral administration, comprising:
a) amiodarone or a pharmaceutically acceptable salt thereof;
b) cyclodextrin; and
c) and a surfactant,
said liquid amiodarone composition having a pH of 3-5.0,
wherein when said liquid amiodarone composition is stored in a pharmaceutically-acceptable container with an amount of an inert gas, at temperature of from about 5-25° C. for a period of at least about 6 months, the amiodarone retains at least about 90% of its starting concentration.

2. The liquid amiodarone composition of claim 1, wherein the amiodarone is amiodarone HCl.

3. The liquid amiodarone composition of claim 1, wherein the cyclodextrin is selected from the group consisting of alpha, gamma, beta cyclodextrin or their derivatives.

4. The liquid amiodarone composition of claim 1, wherein the cyclodextrin is Hydroxy propyl-β-cyclodextrin.

5. The liquid amiodarone composition of claim 1, wherein the amount of cyclodextrin is from about 0.5 to about 5% W/V of the composition.

6. The liquid amiodarone composition of claim 5, wherein the amount of cyclodextrin is from about 1.5 to about 3% W/V of the composition.

7. The liquid amiodarone composition of claim 6, wherein the amount of cyclodextrin is about 2.5% W/V of the composition.

8. The liquid amiodarone composition of claim 1, wherein the surfactant is selected from the group consisting of PEG esters such as DSPE-PEG 2000. PEG-Cholesterol, PEG-Vitamin E. Cremophore, Tweens, solutols and mixtures thereof.

9. The liquid amiodarone composition of claim 8, wherein the surfactant is DSPE-PEG 2000.

10. The liquid amiodarone composition of claim 8, wherein the surfactant is Vitamin E TPGS.

11. The liquid amiodarone composition of claim 1, wherein the amount of surfactant is from about 0.2 to about 0.5% W/V of the composition.

12. The liquid amiodarone composition of claim 11, wherein the amount of surfactant is from about 0.3 to about 0.4% W/V of the composition.

13. The liquid amiodarone composition of claim 1, further comprising a member of the group consisting of antioxidant, non-aqueous solvents, buffering agents, tonicity adjusting agents, pH adjustors, solubilizers, acidic excipients, preservatives and mixtures thereof.

14. The amiodarone composition of claim 13, wherein the acidic excipients are selected from the group consisting of citric acid, succinic acid, methane sulfonic acid, hydrochloric acid, malic acid, malonic acid, maleic acid, tartaric acid, lactic acid, gluconic acid and mixtures thereof.

15. The amiodarone composition of claim 13, wherein the buffering agent is a buffer salts selected from the group consisting of sodium citrate, sodium phosphate, potassium phosphate, sodium acetate and mixtures thereof.

16. The liquid amiodarone composition of claim 1, wherein the composition is packed in a pharmaceutically acceptable container, and the container comprises a copolyester, polyethylene or polyolefin surface in contact with the liquid amiodarone composition.

17. The liquid amiodarone composition of claim 1, wherein the concentration of the amiodarone in the composition is from about 0.1 mg/mL to about 10 mg/mL.

18. The liquid amiodarone composition of claim 17, wherein the concentration of the amiodarone in the composition is from about 1 mg/mL to about 2 mg/mL.

19. The liquid amiodarone composition of claim 13, wherein the tonicity adjusting agent is selected from the group consisting of sodium chloride, dextrose, sorbitol, ringer lactate, mannitol, sucrose, trehalose, amino acids, propylene glycol, glycerol and mixtures thereof.
